(19) United States
(12) Patent Application Publication
(10) Pub. No.: US 2024/0153592 A1
(43) Pub. Date: May 9, 2024

(54) GENE REGULATORY RELATIONSHIP DETECTION MODEL TRAINING METHOD AND APPARATUS AND REGULATORY RELATIONSHIP DETECTION METHOD AND APPARATUS

(71) Applicant: Tencent Technology (Shenzhen) Company Limited, Shenzhen (CN)

(72) Inventors: Fan YANG, Shenzhen (CN); Jiawei LI, Shenzhen (CN); Fang WANG, Shenzhen (CN); Jianhua YAO, Shenzhen (CN)

(21) Appl. No.: 18/415,421

(22) Filed: Jan. 17, 2024

Related U.S. Application Data
(63) Continuation of application No. PCT/CN2023/117427, filed on Sep. 7, 2023.

(30) Foreign Application Priority Data
Oct. 21, 2022 (CN) 202211295954.0

(51) Int. Cl. G16B 40/20

(52) U.S. Cl. CPC G16B 40/20; G06N 7/01

(57) ABSTRACT

A gene regulatory relationship detection model training method performed by an electronic device, and relate to the field of biology technologies. The gene regulatory relationship detection model training method includes: obtaining material group data of a plurality of sample genes and an annotated regulatory relationship between at least one sample gene pair; determining a predicted probability that a regulatory relationship exists between each two sample genes among the plurality of sample genes by using a neural network model based on the material group data of the plurality of sample genes; and training the neural network model to obtain a gene regulatory relationship detection model, based on the annotated regulatory relationship between the at least one sample gene pair and the predicted probability between each two sample genes.

### CROSS-REFERENCE TO RELATED APPLICATIONS

This application is a continuation application of PCT Patent Application No. PCT/CN2023/117427, entitled “GENE REGULATORY RELATIONSHIP DETECTION MODEL TRAINING METHOD AND APPARATUS AND REGULATORY RELATIONSHIP DETECTION METHOD AND APPARATUS” filed on Sep. 7, 2023, which claims priority to Chinese Patent Application No. 202211295954.0, entitled “GENE REGULATORY RELATIONSHIP DETECTION MODEL TRAINING METHOD AND APPARATUS AND REGULATORY RELATIONSHIP DETECTION METHOD AND APPARATUS” filed on Oct. 21, 2022, all of which is incorporated herein by reference in its entirety.

### FIELD OF THE TECHNOLOGY

Embodiments of this application relate to the field of biology technologies, and in particular, to a gene regulatory relationship detection model training method and apparatus and a regulatory relationship detection method and apparatus.

### BACKGROUND OF THE DISCLOSURE

In the field of biology technologies, a gene may direct synthesis of a protein. When a first gene directs synthesis of a protein, and this protein can bind to a specific sequence in a second gene to regulate the second gene to direct synthesis of another protein, a regulatory relationship exists between the first gene and the second gene.

By analyzing a regulatory relationship between genes, a mechanism of disease occurrence and development can be understood at a level of gene regulation, because disease occurrence and development may be related to the synthesis of the protein. Therefore, how to detect the regulatory relationship between the genes has become an urgent problem to be resolved.

### SUMMARY

A gene regulatory relationship detection model training method and apparatus and a regulatory relationship detection method and apparatus are provided in this application, and may be configured to detect a regulatory relationship between genes. The technical solutions include the following content.

According to one aspect, a gene regulatory relationship detection model training method is provided, the method is performed by an electronic device, and the method includes:
obtaining material group data of a plurality of sample genes and an annotated regulatory relationship between at least one sample gene pair indicating that a regulatory relationship exists between two sample genes in the sample gene pair;
determining a predicted probability that a regulatory relationship exists between each two sample genes among the plurality of sample genes by using a neural network model based on the material group data of the plurality of sample genes; and
training the neural network model to obtain a gene regulatory relationship detection model, based on the annotated regulatory relationship between the at least one sample gene pair and the predicted probability between each two sample genes.

According to another aspect, a regulatory relationship detection method is provided, the method is performed by an electronic device, and the method includes:
obtaining material group data of a plurality of target genes and a gene regulatory relationship detection model, the gene regulatory relationship detection model being obtained by training according to the foregoing gene regulatory relationship detection model training method;
determining a predicted probability that a regulatory relationship exists between each two target genes among the plurality of target genes by using the gene regulatory relationship detection model based on the material group data of the plurality of target genes, a predicted probability between any two target genes being a probability that a regulatory relationship exists between the two target genes, obtained by prediction; and
for the two target genes, determining, based on the probability that the regulatory relationship exists between the two target genes, whether the regulatory relationship exists between the two target genes.

According to another aspect, an electronic device is provided, the electronic device includes a processor and a memory, the memory has at least one computer program stored thereon, and the at least one computer program is loaded and executed by the processor to enable the electronic device to implement the foregoing gene regulatory relationship detection model training method or implement the foregoing regulatory relationship detection method.

According to another aspect, a non-transitory computer-readable storage medium is provided, the non-transitory computer-readable storage medium has at least one computer program stored thereon, and the at least one computer program is loaded and executed by a processor to enable an electronic device to implement the foregoing gene regulatory relationship detection model training method or implement the foregoing regulatory relationship detection method.

In the technical solutions provided in this application, a predicted regulatory relationship between each two sample genes among the plurality of sample genes is determined by using the neural network model based on the material group data of the plurality of sample genes, and the neural network model is trained to obtain the gene regulatory relationship detection model, based on the annotated regulatory relationship between the at least one sample gene pair and the predicted regulatory relationship between each two sample genes, so that the gene regulatory relationship detection model can detect whether the regulatory relationship exists between the two target genes, to facilitate understanding a mechanism of disease occurrence and development at a level of gene regulation.

### BRIEF DESCRIPTION OF THE DRAWINGS

FIG. 1 is a schematic diagram of an implementation environment of a gene regulatory relationship detection model training method or a regulatory relationship detection method according to an embodiment of this application.

FIG. 2 is a flowchart of a gene regulatory relationship detection model training method according to an embodiment of this application.

FIG. 3 is a flowchart of a regulatory relationship detection method according to an embodiment of this application.

FIG. 4 is a schematic diagram of detecting a regulatory relationship according to an embodiment of this application.

FIG. 5 is a schematic diagram of a structure of a gene regulatory relationship detection model training apparatus according to an embodiment of this application.

FIG. 6 is a schematic diagram of a structure of a regulatory relationship detection apparatus according to an embodiment of this application.

FIG. 7 is a schematic diagram of a structure of a terminal device according to an embodiment of this application.

FIG. 8 is a schematic diagram of a structure of a server according to an embodiment of this application.

### DESCRIPTION OF EMBODIMENTS

To make the objectives, technical solutions, and advantages of this application clearer, the following further describes implementations of this application in detail with reference to the accompanying drawings.

FIG. 1 is a schematic diagram of an implementation environment of a gene regulatory relationship detection model training method or a regulatory relationship detection method according to an embodiment of this application. As shown in FIG. 1, the implementation environment includes a terminal device 101 and a server 102. The gene regulatory relationship detection model training method or the regulatory relationship detection method in embodiments of this application may be performed by the terminal device 101, may be performed by the server 102, or may be performed by the terminal device 101 and the server 102 jointly.

The terminal device 101 may be a smart phone, a game console, a desktop computer, a tablet computer, a laptop portable computer, a smart TV, a smart vehicle-mounted device, a smart voice interaction device, an intelligent household appliance, and the like. The server 102 may be one server, a server cluster formed by a plurality of servers, or any one of a cloud computing center and a virtualization center. This is not limited in embodiments of this application. The server 102 and the terminal device 101 may perform a communication connection by using a wired network or a wireless network. The server 102 may have functions such as data processing, data storage, and data sending and receiving. This is not limited in embodiments of this application. A quantity of terminal devices 101 and servers 102 is not limited and may be one or more.

The gene regulatory relationship detection model training method or the regulatory relationship detection method provided in embodiments of this application may be implemented based on an artificial intelligence (AI) technology. Artificial intelligence is a theory, a method, a technology, and an application system that use a digital computer or a machine controlled by a digital computer to simulate, extend, and expand human intelligence, perceive an environment, obtain knowledge, and use knowledge to obtain a result. In other words, the artificial intelligence is a comprehensive technology in computer science and attempts to understand the essence of intelligence and produce a new intelligent machine that can react in a manner similar to human intelligence. The artificial intelligence is to study the design principles and implementation methods of various intelligent machines, to enable the machines to have the functions of perception, reasoning, and decision-making.

The artificial intelligence technology is a comprehensive discipline, and relates to a wide range of fields including both hardware-level technologies and software-level technologies. The basic artificial intelligence technologies generally include technologies such as a sensor, a dedicated artificial intelligence chip, cloud computing, distributed storage, a big data processing technology, an operating/interaction system, and electromechanical integration. An artificial intelligence software technology mainly includes some major directions such as a computer vision technology, a speech processing technology, a natural language processing technology, and machine learning/deep learning, automated driving, and smart transportation.

In the field of biology technologies, a regulatory relationship between genes is of vital importance. The regulatory relationship between the genes indicates: When a gene directs synthesis of a protein, and this protein can bind to a specific sequence in another gene to regulate the another gene to direct synthesis of another protein, a regulatory relationship exists between the two genes. By analyzing a regulatory relationship between genes, a mechanism of disease occurrence and development can be understood at a level of gene regulation, because disease occurrence and development may be related to the synthesis of the protein. Therefore, how to detect the regulatory relationship between the genes has become an urgent problem to be resolved.

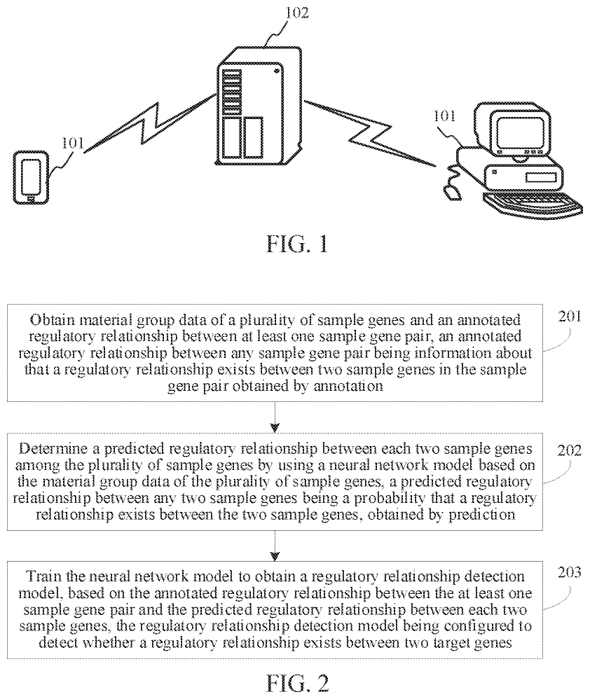

Embodiments of this application provide a gene regulatory relationship detection model training method, and the method may be applied to the foregoing implementation environment. By using this method, a gene regulatory relationship detection model may be obtained by training, and the gene regulatory relationship detection model is used to detect whether a regulatory relationship exists between two target genes. A flowchart of a gene regulatory relationship detection model training method according to an embodiment of this application shown in FIG. 2 is used as an example. A terminal device 101 or a server 102 performing the gene regulatory relationship detection model training method in this embodiment of this application is referred to as an electronic device for ease of description. The method may be performed by the electronic device. As shown in FIG. 2, the method includes the following steps.

Step 201. Obtain material group data of a plurality of sample genes and an annotated regulatory relationship between at least one sample gene pair, an annotated regulatory relationship between any sample gene pair being information about that a regulatory relationship exists between two sample genes in the sample gene pair obtained by annotation.

In other words, obtain material group data of a plurality of sample genes and an annotated regulatory relationship between at least one sample gene pair obtained by annotation, an annotated regulatory relationship between any sample gene pair indicating that a regulatory relationship exists between two sample genes in the sample gene pair. In one embodiment, the electronic device may obtain a sample data set. The sample data set includes the material group data of the plurality of sample genes and the annotated regulatory relationship between the at least one sample gene pair. Material group data of a sample gene and an annotated regulatory relationship between a sample gene pair are both training data for a neural network model, and are used for training the neural network model.

The sample gene may be any gene. A gene is an entire nucleotide sequence needed for producing a peptide chain or a functional ribonucleic acid (RNA). The gene controls expression of a genetic character by directing synthesis of a protein, and a process of directing the synthesis of the protein by the gene may be referred to as a process of gene expression.

The process of the gene expression is usually divided into a transcription process and a translation process. The transcription process is a process of generating an RNA by using the gene as a template. The generated RNA is also referred to as a transcriptome of the gene, and transcriptome data of the gene may be used for describing the transcriptome of the gene. The translation process is a process of generating a protein based on the transcriptome of the gene. The generated protein is also referred to as a proteome of the gene, and proteome data of the gene may be used for describing the proteome of the gene. In embodiments of this application, a material group of the sample gene includes at least one of a transcriptome of the sample gene and a proteome of the sample gene. In other words, the material group data of the sample gene includes at least one of transcriptome data of the sample gene and proteome data of the sample gene.

In one embodiment, the transcriptome of the gene is extracted from a cell or a tissue. By sequencing the transcriptome of the gene, data obtained by sequencing may be used as transcriptome data of the gene or a part of transcriptome data of the gene. Alternatively, a quantity of RNAs produced by using the gene as the template may be measured in the cell or the tissue. The quantity of RNAs is an expression value of the gene in the cell (because materials used for making up the tissue include the cell, even when the measurement is performed in the tissue, the quantity of RNAs may also be referred to as the expression value of the gene in the cell). The expression value of the gene in the cell is used as transcriptome data of the gene or a part of transcriptome data of the gene.

Similarly, the proteome of the gene is extracted from the cell or the tissue. By sequencing the proteome of the gene, data obtained by sequencing may be used as proteome data of the gene or a part of proteome data of the gene. Alternatively, a quantity of proteins produced based on the gene may be measured in the cell or the tissue. The quantity of proteins is also an expression value of the gene in the cell (because the materials used for making up the tissue include the cell, even when the measurement is performed in the tissue, the quantity of proteins may also be referred to as the expression value of the gene in the cell). The expression value of the gene in the cell is used as transcriptome data of the gene or a part of transcriptome data of the gene.

The foregoing mentioned cell may be unicellular or multicellular, and may be any type of cell, such as a brain cell or a skin cell. The tissue is a substance including a group of cells and intercellular substances having similar forms and same functions, and may be a tissue of a clinical sample. The expression value of the gene in the cell may be a sum of the quantity of RNAs produced by using the gene as the template and the quantity of proteins produced based on the gene. The material group data of the plurality of sample genes may be recorded by using a matrix. One row of data in the matrix is material group data of one sample gene.

Any two sample genes may be considered as one sample gene pair. When a regulatory relationship exists between two sample genes in any sample gene pair, the annotated regulatory relationship between the sample gene pair may be obtained by annotating the sample gene pair. For example, whether a regulatory relationship exists between two sample genes is manually determined. In response to determining that the regulatory relationship exists, the two sample genes are annotated to obtain an annotated regulatory relationship between a sample gene pair formed by the two sample genes. Alternatively, a reported regulatory relationship between genes included in a gene regulatory network (GRN) database may be collected. When a regulatory relationship between two sample genes is reported, the two sample genes are annotated to obtain an annotated regulatory relationship between a sample gene pair formed by the two sample genes.

The GRN database includes, but is not limited to: a regulatory network (RegNetwork) database, a Kyoto Encyclopedia of Genes and Genomes (KEGG) database, a Reactome database, a Coessentiality database, and the like.

Step 202. Determine a predicted regulatory relationship between each two sample genes among the plurality of sample genes by using the neural network model based on the material group data of the plurality of sample genes, a predicted regulatory relationship between any two sample genes being a probability that a regulatory relationship exists between the two sample genes, obtained by prediction.

The predicted regulatory relationship may also be referred to as a predicted probability. A predicted probability between any two sample genes is a probability that a regulatory relationship exists between the two sample genes, obtained by prediction. The material group data of the plurality of sample genes may be inputted into the neural network model. A probability that a regulatory relationship exists between each two sample genes among the plurality of sample genes may be outputted by the neural network model. The probability that the regulatory relationship exists between the two sample genes is data that is greater than or equal to 0 and less than or equal to 1. A greater probability that the regulatory relationship exists between the two sample genes indicates that the regulatory relationship is more likely to exist between the two sample genes.

The neural network model in this embodiment of this application may be an untrained initial network model. In this case, a model structure, a model size, and the like of the neural network model are the same as a model structure, a model size, and the like of the initial network model. The model structure, model size, and the like of the initial network model are not limited in this embodiment of this application. For example, the initial network model includes an encoder and a decoder. For functions of the encoder and functions of the decoder, refer to related description below. Details are not described herein again. In one embodiment, the neural network model may alternatively be a model obtained by training the initial network model at least once according to step 201 to step 203, or a model obtained by training the initial network model at least once according to another training method.

In a possible implementation, step 202 includes step 2021 and step 2022.

Step 2021. Perform feature extraction on the material group data of the plurality of sample genes by using the neural network model based on the annotated regulatory relationship between the at least one sample gene pair, to obtain a gene feature of each sample gene.

For any sample gene, the encoder included in the neural network model may perform feature extraction on material group data of the sample gene based on the annotated regulatory relationship between the at least one sample gene pair, to obtain a gene feature of the sample gene.

A structure, a size, and the like of the encoder are not limited in this embodiment of this application. For example, the encoder may be convolutional neural network (CNN), and the encoder includes at least one convolution block. Input of the first convolution block includes the annotated regulatory relationship between the at least one sample gene pair and material group data of any sample gene, and output of the last convolution block includes a gene feature of any sample gene. A previous convolution block is connected to a next convolution block in series, so that input of the next convolution block includes output of the previous convolution block (which may be denoted as first input). In addition, the input of the next convolution block further includes the annotated regulatory relationship between the at least one sample gene pair (which may be denoted as second input). Any convolution block may perform convolution processing on first input of the convolution block based on second input of the convolution block, to reduce a data dimension of the first input and obtain output of the convolution block.

In an implementation A, step 2021 includes step A1 and step A2.

Step A1. For any sample gene, determine a gene feature of the sample gene by using the neural network model based on material group data of the sample gene and material group data of an adjacent gene of the sample gene, when it is determined, based on the annotated regulatory relationship between the at least one sample gene pair, that the adjacent gene of the sample gene exists, the adjacent gene of the sample gene being a sample gene that has a regulatory relationship with the sample gene.

Any sample gene may have at least one adjacent gene. The encoder may determine the gene feature of the sample gene based on the material group data of the sample gene and material group data of each adjacent gene, so that the gene feature of the sample gene may represent material group data of a sample gene that has a regulatory relationship with the sample gene other than represent the material group data of the sample gene, thereby improving a representing capability of the gene feature of the sample gene. By extracting the gene feature of the sample gene, the material group data of the sample gene can be smoothed to a certain extent and noise can be reduced. When the gene feature of the sample gene is subsequently used for determining a predicted regulatory relationship between two sample genes, accuracy of the predicted regulatory relationship may be improved, so that accuracy of the model can be improved when the predicted regulatory relationship is used for training the model.

In this embodiment of this application, when a regulatory relationship exists between two sample genes in any sample gene pair, the encoder may match the sample gene pair with any sample gene. When the sample gene pair includes the sample gene, the other sample gene included in the sample gene pair is an adjacent gene of the sample gene.

For example, a sample gene pair includes a sample gene A and a sample gene B, and a regulatory relationship exists between the sample gene A and the sample gene B. The encoder matches the sample gene pair with a sample gene m. When the sample gene A is the sample gene m, the sample gene B is an adjacent gene of the sample gene m. When the sample gene B is the sample gene m, the sample gene A is an adjacent gene of the sample gene m.

By using the foregoing manner, an adjacent gene of any sample gene may be determined based on the annotated regulatory relationship between the at least one sample gene pair. Any sample gene corresponds to at least one adjacent gene, and the encoder may splice material group data of the sample gene with material group data of each adjacent gene to obtain spliced data. By performing feature extraction on the spliced data, a gene feature of the sample gene is obtained.

In one embodiment, the encoder includes at least one convolution block. Because a convolution block is used for convolution processing, output of a k^{th} (where k is a positive integer) convolution block is denoted as a k^{th} convolution processing result for ease of description. In this embodiment of this application, input of the first convolution block includes spliced data obtained by splicing material group data of any sample gene with material group data of each adjacent gene of the sample gene. The first convolution block performs convolution processing on the spliced data, to reduce a data dimension of the spliced data, thereby obtaining and outputting a first convolution processing result of the sample gene. Input of an i^{th} convolution block (where i is a positive integer greater than or equal to 2) includes a spliced result obtained by splicing an (i−1)^{th} convolution processing result of the sample gene with an (i−1)^{th} convolution processing result of each adjacent gene of the sample gene. The i^{th} convolution block performs convolution processing on the spliced result, to reduce a data dimension of the spliced result, thereby obtaining and outputting an i^{th} convolution processing result of the sample gene. The last convolution processing result that is of any sample gene and that is outputted by the last convolution block is a gene feature of the sample gene.

Step A2. Determine a gene feature of the sample gene by using the neural network model based on material group data of the sample gene, when it is determined, based on the annotated regulatory relationship between the at least one sample gene pair, that no adjacent gene of the sample gene exists.

In this embodiment of this application, the annotated regulatory relationship between the sample gene pair is used for describing a regulatory relationship existing between two sample genes in the sample gene pair. In other words, the regulatory relationship exists between the sample gene pair. In application, the encoder may match the sample gene pair having the regulatory relationship with any sample gene. When the sample gene pair having the regulatory relationship does not include the sample gene, then no adjacent gene of the sample gene exists.

For example, one sample gene pair having a regulatory relationship includes a sample gene A and a sample gene B (in other words, a regulatory relationship exists between the sample gene A and the sample gene B), and another sample gene pair having a regulatory relationship includes a sample gene C and a sample gene D (in other words, a regulatory relationship exists between the sample gene C and the sample gene D). However, sample genes A to D are different from a sample gene n. In other words, the two sample gene pairs having the regulatory relationship do not include the sample gene n. In this case, no adjacent gene of the sample gene n exists.

When no adjacent gene of a sample gene exists, the encoder may perform feature extraction on material group data of the sample gene, to obtain a gene feature of the sample gene. In one embodiment, the encoder includes at least one convolution block. Output of a k^{th} (where k is a positive integer) convolution block is denoted as a k^{th} convolution processing result for ease of description.

In this embodiment of this application, input of the first convolution block includes material group data of any sample gene. The first convolution block performs convolution processing on the material group data of the sample gene to reduce a data dimension of the material group data of the sample gene, thereby obtaining and outputting a first convolution processing result of the sample gene. Input of an i^{th} convolution block (where i is a positive integer greater than or equal to 2) includes an (i−1)^{th} convolution processing result of the sample gene. The i^{th} convolution block performs convolution processing on the (i−1)^{th} convolution processing result of the sample gene, to reduce a data dimension of the (i−1)^{th} convolution processing result of the sample gene, thereby obtaining and outputting an i^{th} convolution processing result of the sample gene. The last convolution processing result that is of any sample gene and that is outputted by the last convolution block is a gene feature of the sample gene.

Foregoing step A1 and step A2 both include a process in which the encoder matches the sample gene pair having the regulatory relationship with any sample gene. It is to be understood that when there is one sample gene pair, the encoder matches the sample gene pair with any sample gene to determine whether at least one adjacent gene of the sample gene exists. When there are a plurality of sample gene pairs, after each sample gene pair is matched with any sample gene, that is, after the plurality of sample gene pairs are matched, the encoder determines whether at least one adjacent gene of the sample gene exists.

In one embodiment, any sample gene has unique identification information. By matching two pieces of identification information of a sample gene pair with identification information of any sample gene, the encoder matches the sample gene pair with the sample gene. Compared with a method of directly matching the sample gene itself, a method of matching the identification information of the sample gene is more concise and efficient, thereby facilitating improving efficiency of model training.

In an implementation B, step 2021 includes step B1 to step B3.

Step B1. Determine each sample node based on material group data of each sample gene, one sample node representing material group data of one sample gene.

Material group data of one sample gene may be used as one sample node. Alternatively, feature extraction may be performed on material group data of one sample gene to obtain an initial feature of the sample gene, and the initial feature of the sample gene may be used as one sample node. A method for feature extraction is not limited in this embodiment of this application. For example, principal component analysis is performed on material group data of one sample gene, and extracted principal component data is used as an initial feature of the sample gene. Alternatively, by counting material group data of a sample gene, counted data is used as an initial feature of the sample gene.

In this embodiment of this application, one sample node corresponds to one sample gene. Therefore, a quantity of sample nodes is the same as a quantity of sample genes.

Step B2. For any two sample nodes, add a sample edge between the two sample nodes, when it is determined, based on the annotated regulatory relationship between the at least one sample gene pair, that a regulatory relationship exists between sample genes corresponding to the two sample nodes.

In this embodiment of this application, the annotated regulatory relationship between any sample gene pair is used for describing a regulatory relationship existing between the sample gene pair. For a sample gene pair having a regulatory relationship, the regulatory relationship exists between two sample genes included in the sample gene pair. A sample edge may be added between sample nodes corresponding to the two sample genes included in the sample gene pair. It is to be understood that when no regulatory relationship exists between the two sample genes, no sample edge exists between the sample nodes corresponding to the two sample genes.

By determining each sample node and determining, based on the annotated regulatory relationship between the at least one sample gene pair, whether a sample edge is added between each two sample nodes, a sample gene map including a plurality of sample nodes and at least one sample edge may be obtained, so that sample nodes of sample genes having a regulatory relationship are connected in the sample gene map. The sample gene map is a type of graph-structured data. The sample gene map can not only reflect material group data of a single sample gene but also reflect information about whether a regulatory relationship exists between each two sample genes. The sample gene map is constructed by using the annotated regulatory relationship of the at least one sample gene pair, so that a topological structure of the sample gene map is determined by using biological prior knowledge, a logic of the material group data of the sample gene is achieved, and input of the encoder is improved, to facilitate improving accuracy of an output result of the encoder.

Step B3. Perform feature extraction on the sample gene map by using the neural network model to obtain the gene feature of each sample gene, the sample gene map including each sample node and the sample edge.

A graph structure may include each sample node and each sample edge, and the graph structure is denoted as the sample gene map. The encoder included in the neural network model may perform feature extraction on each sample node in the sample gene map to obtain a feature of each sample node. A feature of any sample node is a gene feature of a sample gene corresponding to the sample node.

In this embodiment of this application, the encoder may include any one of a graph convolutional network (GCN), a graph attention network (GAT), a graph transformer, a molecular structure model (such as Graphormer), and the like.

For example, the encoder includes at least one layer of the GCN. Any layer of the GCN is configured to perform graph convolution processing on an inputted graph structure, to update each sample node in the graph structure, and obtain and output an updated graph structure. For ease of description, an outputted graph structure of a k^{th} (where k is a positive integer) layer of the GCN is denoted as a k^{th} graph structure.

In this embodiment of this application, the sample gene map is one graph structure. Input of the first layer of the GCN is the sample gene map, and the first layer of the GCN is configured to perform graph convolution processing on the sample gene map to update each sample node in the sample gene map. An updated sample gene map is used as a first graph structure, and the first graph structure is outputted. A previous layer of the GCN is connected to a next layer of the GCN in series, so that input of the next layer of the GCN includes output of the previous layer of the GCN. Any layer of the GCN may perform graph convolution processing on a graph structure inputted to the layer of the GCN, to update a sample node in the graph structure, and obtain an updated graph structure and output the updated graph structure. Output of the last layer of the GCN is the last graph structure, and each sample node in the last graph structure is a gene feature of each sample gene.

In one embodiment, step B3 includes step B31 and step B32.

Step B31. For any sample node, determine a gene feature of a sample gene corresponding to the sample node based on the sample node and an adjacent node of the sample node, when it is determined, based on the sample gene map, that the adjacent node of the sample node exists, the adjacent node of the sample node being a sample node that has a sample edge with the sample node.

For any sample node in the sample gene map, when a sample node that has a sample edge with the sample node exists in the sample gene map, the existing sample node is an adjacent node of the sample node. For example, in the sample gene map, a sample edge exists between a sample node A and a sample node B. Therefore, the sample node A is an adjacent node of the sample node B, and the sample node B is an adjacent node of the sample node A.

Any sample node has at least one adjacent node. The encoder may update the sample node based on the sample node and each adjacent node, so that the sample node may represent material group data of a sample gene that has a regulatory relationship with the sample gene corresponding to the sample node other than represent the material group data of the sample gene corresponding to the sample node itself, thereby improving a representing capability of the sample node.

In one embodiment, the encoder includes at least one layer of the GCN. Input of the first layer of the GCN includes the sample gene map. For any sample node in the sample gene map, the first layer of the GCN uses a sample node that has a sample edge with the sample node in the sample gene map as an adjacent node of the sample node. By performing graph convolution processing on the sample node and each adjacent node, the sample node may be updated. In this way, each sample node in the sample gene map is updated, and an updated sample gene map is used as the first graph structure. Input of an i^{th} (where i is a positive integer greater than or equal to 2) layer of the GCN includes an (i−1)^{th} graph structure. For any sample node in the (i−1)^{th} graph structure, the i^{th} layer of the GCN uses a sample node that has a sample edge with the sample node in the (i−1)^{th} graph structure as an adjacent node of the sample node. By performing graph convolution processing on the sample node and each adjacent node, the sample node may be updated. In this way, each sample node in the (i−1)^{th} graph structure is updated, and an updated sample gene map is used as the i^{th} graph structure. Each sample node in the last graph structure outputted by the last layer of the CGN is a gene feature of each sample gene.

Step B32. Determine a gene feature of a sample gene corresponding to the sample node based on the sample node, when it is determined, based on the sample gene map, that no adjacent node of the sample node exists.

For any sample node in the sample gene map, when no sample node that has a sample edge with the sample node may exist in the sample gene map, no adjacent node of the sample node exists. For example, in the sample gene map, a sample node A is an isolated node, and no sample edge exists between the sample node A and another sample node in the sample gene map. In this case, no adjacent node of the sample node A exists.

When no adjacent node of any sample node exists, the encoder may update the sample node based on the sample node. In one embodiment, the encoder includes at least one layer of the GCN. Input of the first layer of the GCN includes the sample gene map. For any sample node in the sample gene map, when no adjacent node of the sample node exists, by performing graph convolution processing on the sample node, the sample node may be updated. In this way, each sample node in the sample gene map is updated, and an updated sample gene map is used as the first graph structure. Input of an i^{th} (where i is a positive integer greater than or equal to 2) layer of the GCN includes an (i−1)^{th} graph structure. For any sample node in the (i−1)^{th} graph structure, when no adjacent node of the sample node exists, by performing graph convolution processing on the sample node, the sample node may be updated. In this way, each sample node in the (i−1)^{th} graph structure is updated, and an updated sample gene map is used as the i^{th} graph structure. Each sample node in the last graph structure outputted by the last layer of the CGN is a gene feature of each sample gene.

Step 2022. Determine the predicted regulatory relationship (that is, the predicted probability) between each two sample genes by using the neural network model based on the gene feature of each sample gene.

The decoder included in the neural network model may determine, based on the gene feature of each sample gene, the predicted regulatory relationship between each two sample genes. A structure, a size, and the like of the decoder are not limited in this embodiment of this application. For example, the decoder is a transformer-based decoder. The decoder may include a fully-connected network and an activation network, and the fully-connected network is at least one layer. For functions of the fully-connected network and the activation network, refer to related description below. Details are not described herein again.

In one embodiment, step 2022 includes step C1 and step C2.

Step C1. For any two sample genes, determine a degree of similarity between gene features of the two sample genes by using the neural network model.

The fully-connected network included in the decoder may calculate a degree of similarity between the gene features of any two sample genes based on the gene features of the two sample genes. Alternatively, the decoder may first perform dimension reduction on a gene feature of each sample gene, and then calculates a degree of similarity between gene features of any two sample genes after the dimension reduction is performed. A method of calculating the degree of similarity is not limited in this embodiment of this application. For example, gene features of two sample genes are multiplied, and an obtained product result is used as a degree of similarity between the gene features of the two sample genes. Alternatively, a cosine value of an angle between gene features of two sample genes is calculated and the cosine value of the angle is used as a degree of similarity between the gene features of the two sample genes.

Step C2. Determine the predicted regulatory relationship (that is, the predicted probability) between the two sample genes based on the degree of similarity between the gene features of the two sample genes.

The activation network included in the decoder may activate the degree of similarity between the gene features of the two sample genes. The degree of similarity is converted, by performing activation processing, into a probability that the regulatory relationship exists between the two sample genes, to obtain the predicted regulatory relationship between the two sample genes. In one embodiment, the activation network is an S-shaped curve (that is, a sigmoid function), a rectified linear unit (ReLU), or the like. The activation network may map input (that is, input of the activation network) to a probability. In this embodiment of this application, the input of the activation network is the degree of similarity between the gene features of the two sample genes, and the probability obtained by mapping is the predicted regulatory relationship between the two sample genes.

In another possible implementation, step 202 includes step 2023 to step 2025.

Step 2023. For the two sample genes, determine a sample scatter plot based on material group data of the two sample genes, material group data of any sample gene including expression values of the sample gene in a plurality of cells, the sample scatter plot including a plurality of sample points, and any sample point representing expression values of the two sample genes in a same cell.

In this embodiment of this application, an expression value of a sample gene in a cell is used for measuring an expression level of the sample gene in the cell. As mentioned above, the products expressed by the sample gene include the material group of the sample gene. Therefore, the expression value of the sample gene in the cell may measure a size of the material group of the sample gene. A greater quantity of materials included in the material group of the sample gene indicates a larger material group of the sample gene and a higher expression value of the sample gene in the cell. For example, more proteins expressed by the sample gene indicate a larger proteome of the sample gene and a higher expression value of the sample gene in the cell.

A scatter plot may be generated by using the material group data of the two sample genes. Because the material group data of the sample gene includes expression values of the sample gene in a plurality of cells, expression values of the two sample genes in the same cell may be regarded as a horizontal coordinate and a vertical coordinate respectively, and a sample point in the sample scatter plot may be determined based on the horizontal coordinate and the vertical coordinate. One sample point corresponds to one cell. Therefore, a quantity of sample points is equal to a quantity of cells.

For example, material group data of a sample gene A includes expression values of the sample gene A in a cell 1 to a cell 100, and material group data of a sample gene B includes expression values of the sample gene B in a cell 1 to a cell 100. In this case, an expression value of the sample gene A in the cell 1 may be regarded as a horizontal coordinate of a sample point corresponding to the cell 1, and an expression value of the sample gene B in the cell 1 may be regarded as a vertical coordinate of the sample point corresponding to the cell 1, to determine a location of the sample point corresponding to the cell 1. Similarly, an expression value of the sample gene A in the cell 2 may be regarded as a horizontal coordinate of a sample point corresponding to the cell 2, and an expression value of the sample gene B in the cell 2 may be regarded as a vertical coordinate of the sample point corresponding to the cell 2, to determine a location of the sample point corresponding to the cell 2. By analogy, positions of sample points corresponding to the cell 1 to the cell 100 may be determined, to obtain the sample scatter plot.

Step 2024. Perform feature extraction on the sample scatter plot by using the neural network model to obtain sample image features.

The sample scatter plot is an image. Therefore, the sample scatter plot includes a plurality of pixel points. A pixel value of any pixel point may be first data or may be second data. When the pixel value of the pixel point is the first data, it indicates that the pixel point belongs to a certain sample point in the sample scatter plot. When the pixel value of the pixel point is the second data, it indicates that the pixel point does not belong to any sample point in the sample scatter plot.

The encoder included in the neural network model may perform feature extraction on the sample scatter plot based on a pixel value of each pixel point in the sample scatter plot to obtain the sample image features. A method for feature extraction is not limited in this embodiment of this application. For example, the method for feature extraction may be a method such as a scale-invariant feature transform (SIFT) method or a histogram of oriented gradient (HOG) method.

In one embodiment, step 2024 includes: dividing the sample scatter plot into a plurality of sample image regions; and performing the feature extraction on the sample scatter plot by using the neural network model based on a quantity of sample points in each sample image region to obtain the sample image features.

The sample scatter plot may be divided into a set quantity of sample image regions. For example, the sample scatter plot is divided into nine sample image regions. Alternatively, the sample scatter plot may be divided into a plurality of sample image regions having the same size as that of a set window based on the set window. For example, the sample scatter plot is divided into a plurality of sample image regions of 16×16.

Then, for any sample image region, a quantity of sample points included in the sample image region is counted. In a possible implementation, a quantity of sample points included in a sample image region is used as a piece of data of an input matrix. In this way, the input matrix may be determined. A quantity of pieces of data in the input matrix is the same as a quantity of sample image regions. For example, when the sample scatter plot is divided into nine sample image regions, and a quantity of sample points included in the nine sample image regions are 14, 18, 10, 21, 13, 14, 12, 17, and 15 respectively, the input matrix may be

$$[ \begin{matrix}14 & 18 & 10 \\ 21 & 13 & 14 \\ 12 & 17 & 15\end{matrix} ] .$$

In another possible implementation, a ratio of a quantity of sample points included in a sample image region to a size of the sample image region is calculated, and the obtained ratio is used as a piece of data of an input matrix. In this way, the input matrix may be determined. A quantity of pieces of data in the input matrix is the same as a quantity of sample image regions. Because a ratio of a quantity of sample points included in a sample image region to a size of the sample image region may represent a sample point density of the sample image region, any piece of data in the input matrix represents a sample point density of a sample image region corresponding to the data.

The encoder included in the neural network model performs dimension reduction on the input matrix to use a matrix as the sample image feature after the dimension reduction is performed. A process of determining the input matrix based on the sample scatter plot and performing dimension reduction on the input matrix is equivalent to performing feature extraction on the sample scatter plot.

Step 2025. Classify the sample image features by using the neural network model, to obtain the predicted regulatory relationship (that is, the predicted probability) between the two sample genes.

The decoder included in the neural network model may classify the sample image features to map the sample image features to a probability. The probability is the predicted regulatory relationship between the two sample genes. The probability may be regarded as a classification result obtained by classification. In other words, the sample image features is classified by using the neural network model, and the classification result is used as the predicted probability between the two sample genes. In one embodiment, the decoder includes the activation network. The sample image features are inputted into the activation network. The sample image features are mapped to the predicted regulatory relationship between the two sample genes by using the activation network.

In this embodiment of this application, two sample genes are used as a group, one sample gene group determines one sample scatter plot, and a predicted regulatory relationship of the sample gene group is determined based on the sample scatter plot. Therefore, when predicted regulatory relationships between a plurality of sample gene groups needs to be determined, a sample scatter plot corresponding to each group needs to be generated. A predicted regulatory relationship between each sample gene group is determined based on the sample scatter plot corresponding to each group.

Step 203. Train the neural network model to obtain a gene regulatory relationship detection model, based on the annotated regulatory relationship between the at least one sample gene pair and the predicted regulatory relationship between each two sample genes, the gene regulatory relationship detection model being configured to detect whether a regulatory relationship exists between two target genes.

As described above, the predicted regulatory relationship is the predicted probability. Therefore, the neural network model is trained to obtain the gene regulatory relationship detection model, based on the annotated regulatory relationship between the at least one sample gene pair and the predicted probability between each two sample genes. For example, a loss value of the neural network model may be calculated according to a calculation formula of a loss function and based on the annotated regulatory relationship between the at least one sample gene pair and the predicted regulatory relationship between each two sample genes. The loss function is not limited in this embodiment of this application. For example, the loss function is a cross entropy function, a mean absolute error (MAE) function, a mean squared error (MSE) function, or the like.

Then, the neural network model is trained based on the loss value of the neural network model, to obtain a trained neural network model. A gradient descent manner may be used for training the neural network model. To be specific, a gradient of the loss value of the neural network model to model parameters of the neural network model is calculated first, and the model parameters of the neural network model is adjusted along an opposite direction of the gradient, to obtain the trained neural network model.

When the trained neural network model satisfies a training end condition, the trained neural network model is used as the gene regulatory relationship detection model. When the trained neural network model does not satisfy the training end condition, the trained neural network model is used as a neural network model for next training. The next training for the neural network model is performed according to the foregoing step 201 to step 203 until a trained neural network model satisfies the training end condition. The trained neural network model is used as the gene regulatory relationship detection model.

That the training end condition is satisfied is not limited in embodiments of this application. For example, that the training end condition is satisfied means that times of training the neural network model reach set times. For example, the times of training the neural network model reach 500 times. Alternatively, that the training end condition is satisfied means that a difference between a loss value of a neural network model corresponding to current training and a loss value of a neural network model corresponding to the last training is within a set range. For example, the difference between the loss value of the neural network model corresponding to the current training and the loss value of the neural network model corresponding to the last training is 0.003 that is within the set range [−0.002, 0.0033].

In one embodiment, step 203 includes step 2031 and step 2032.

Step 2031. For the two sample genes, determine a reference regulatory relationship between the two sample genes based on the predicted regulatory relationship between the two sample genes, the reference regulatory relationship between the two sample genes being information about whether the regulatory relationship exists between the two sample genes obtained by prediction.

In other words, for the two sample genes, obtain a reference regulatory relationship between the two sample genes based on the predicted probability between the two sample genes by prediction, the reference regulatory relationship between the two sample genes indicating whether the regulatory relationship exists between the two sample genes. In this embodiment of this application, the predicted regulatory relationship between the two sample genes is a probability that the regulatory relationship exists between the two sample genes, obtained by prediction. When the probability that the regulatory relationship exists between the two sample genes is greater than a threshold, it is determined that the regulatory relationship exists between the two sample genes. When the probability that the regulatory relationship exists between the two sample genes is not greater than a threshold, it is determined that no regulatory relationship exists between the two sample genes. The predicted regulatory relationship between the two sample genes is binarized by using the threshold, so that the information about whether the regulatory relationship exists between the two sample genes is obtained.

A value of the threshold is not limited in this embodiment of this application. For example, the value of the threshold is a set value. For example, the threshold is 0.7. Alternatively, a probability that a regulatory relationship exists between each two sample genes is ranked, and a ranked (reference number)^{th} probability is used as the threshold. For example, the ranked tenth probability is used as the threshold.

Step 2032. Train the neural network model to obtain the gene regulatory relationship detection model, based on the annotated regulatory relationship between the at least one sample gene pair and the reference regulatory relationship between the two sample genes.

The reference regulatory relationship between the two sample genes is used for representing whether the regulatory relationship exists between the two sample genes. The annotated regulatory relationship between the sample gene pair is used for representing whether the regulatory relationship exists between two sample genes in the sample gene pair. Third data (for example, 1) may be used for indicating that the regulatory relationship exists between the two sample genes, and fourth data (for example, 0) may be used for indicating that no regulatory relationship exists between the two sample genes. By using the third data or the fourth data, an annotated regulatory relationship between any sample gene pair is represented. In addition, by using the third data or the fourth data, a reference regulatory relationship between any two sample genes is represented. A loss value based on the neural network model may be calculated according to a calculation formula of a loss function and based on the third data and the fourth data. Therefore, by using the loss value of the neural network model, the neural network model is trained, to obtain the gene regulatory relationship detection model.

By using step 201 to step 203, the neural network model is trained automatically, to reduce costs and waiting time for manual analysis, so as to prevent an error problem caused by the manual analysis.

As mentioned above, the neural network model in this embodiment of this application includes the encoder and the decoder. The encoder may perform feature extraction on the material group data of the sample gene to obtain the gene feature of the sample gene. The decoder may determine, based on the gene feature of each sample gene, the predicted regulatory relationship between each two sample genes. The material group data of the sample gene includes transcriptome data of the sample gene and/or proteome data of the sample gene.

When the material group data of the sample gene includes the transcriptome data of the sample gene and the proteome data of the sample gene, in a possible implementation, the neural network model includes two encoders and one decoder. One encoder is used for performing feature extraction on the transcriptome data of the sample gene to obtain a transcriptome feature of the sample gene. The other encoder is used for performing feature extraction on the proteome data of the sample gene to obtain a proteome feature of the sample gene. A transcriptome feature of a sample gene is spliced with a proteome feature of the sample gene to obtain a gene feature of the sample gene. The decoder determines, based on the gene feature of each sample gene, a predicted regulatory relationship between each two sample genes. A method for the encoder to determine a transcriptome feature of a sample gene and a method for the encoder to determine a proteome feature of the sample gene are similar to the method for the encoder to determine the gene feature of the sample gene in step 2021. Details are not described herein again.

Information (including but not limited to user equipment information, user personal information, and the like), data (including but not limited to data used for analysis, stored data, displayed data, and the like), and a signal included in this application are authorized by a user or fully authorized by parties. Collection, use and processing of relevant data shall comply with relevant laws, regulations, and standards of relevant countries and regions. For example, the material group data of the sample gene and the annotated regulatory relationship between the sample gene pair included in this application are obtained under full authorization.

In the foregoing method, a predicted regulatory relationship between each two sample genes among the plurality of sample genes is determined by using the neural network model based on the material group data of the plurality of sample genes. In addition, the neural network model is trained to obtain the gene regulatory relationship detection model, based on the annotated regulatory relationship between the at least one sample gene pair and the predicted regulatory relationship between each two sample genes, so that the gene regulatory relationship detection model can detect whether the regulatory relationship exists between the two target genes, to facilitate understanding a mechanism of disease occurrence and development at a level of gene regulation.

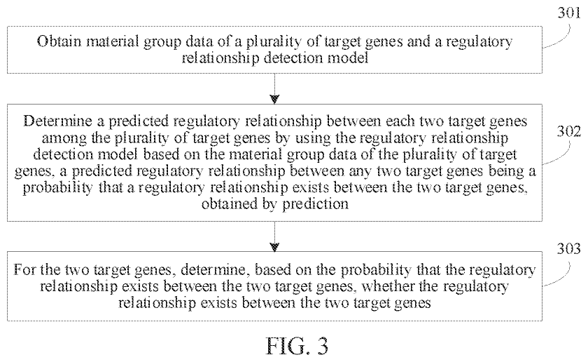

Embodiments of this application provide a regulatory relationship detection method, and the method may be applied to the foregoing implementation environment. In the method, the gene regulatory relationship detection model is used for detecting whether a regulatory relationship exists between two target genes. A flowchart of a regulatory relationship detection method according to an embodiment of this application shown in FIG. 3 is used as an example. A terminal device 101 or a server 102 performing the regulatory relationship detection method in embodiments of this application is referred to as an electronic device for ease of description. The method may be performed by the electronic device. As shown in FIG. 3, the method includes the following steps.

Step 301. Obtain material group data of a plurality of target genes and a gene regulatory relationship detection model.

For the material group data of the target genes, refer to the foregoing description related to the material group data of the sample genes. The gene regulatory relationship detection model is obtained by training according to the gene regulatory relationship detection model training method related to FIG. 2, and refer to the foregoing description of step 201 to step 203. Details are not described herein again.

Step 302. Determine a predicted regulatory relationship between each two target genes among the plurality of target genes by using the gene regulatory relationship detection model based on the material group data of the plurality of target genes, a predicted regulatory relationship between any two target genes being a probability that a regulatory relationship exists between the two target genes, obtained by prediction.

The predicted regulatory relationship is also referred to as a predicted probability. A predicted probability between any two target genes is a probability that a regulatory relationship exists between the two target genes, obtained by prediction. For description of step 302, refer to the description of step 202. Implementation principles of the two are similar. Details are not described herein again.

In an implementation C, step 302 includes: obtaining an annotated regulatory relationship between at least one target gene pair, an annotated regulatory relationship between any target gene pair being information about that a regulatory relationship exists between two target genes in the target gene pair obtained by annotation (in other words, obtaining an annotated regulatory relationship between at least one target gene pair obtained by annotation, an annotated regulatory relationship between any target gene pair indicating that a regulatory relationship exists between two target genes in the target gene pair); performing feature extraction on the material group data of the plurality of target genes by using the gene regulatory relationship detection model based on the annotated regulatory relationship between the at least one target gene pair, to obtain a gene feature of each target gene; and determining the predicted regulatory relationship (that is, the predicted probability) between the two target genes by using the gene regulatory relationship detection model based on the gene feature of each target gene.

For description of the implementation C, refer to the description of step 201, step 2021 and step 2022. Implementation principles of the two are similar Details are not described herein gain.

In an implementation D, step 302 includes: for the two target genes, determining a target scatter plot based on material group data of the two target genes, material group data of any target gene including expression values of the target gene in a plurality of cells, the target scatter plot including a plurality of target points, and any target point representing expression values of the two target genes in a same cell; performing feature extraction on the target scatter plot by using the gene regulatory relationship detection model to obtain target image features; and classifying the target image features by using the gene regulatory relationship detection model to obtain the predicted regulatory relationship (that is, the predicted probability) between the two target genes.

For description of the implementation D, refer to the description of step 2023 to step 2025. Implementation principles of the two are similar Details are not described herein again.

Step 303. For the two target genes, determine, based on the probability that the regulatory relationship exists between the two target genes, whether the regulatory relationship exists between the two target genes.

In one embodiment, step 303 includes: determining, when the probability that the regulatory relationship exists between the two target genes is greater than a threshold, that the regulatory relationship exists between the two target genes; or determining, when the probability that the regulatory relationship exists between the two target genes is not greater than a threshold, that no regulatory relationship exists between the two target genes.

A value of the threshold is not limited in this embodiment of this application. For example, the value of the threshold is a set value. For example, the threshold is 0.7. Alternatively, a probability that a regulatory relationship exists between each two target genes is ranked, and a ranked (reference number)′ probability is used as the threshold. For example, the ranked tenth probability is used as the threshold. For description of step 303, refer to the description of step 2031. Implementation principles of the two are similar Details are not described herein again.

After determining whether the regulatory relationship exists between the two target genes, material group data of each target gene or an initial feature of each target gene may be used as each node in a regulatory relationship network between target genes. An edge is added between nodes of the two target genes having the regulatory relationship, to obtain the regulatory relationship network between the target genes. When there is a differential gene in the target genes, a target gene that has a regulatory relationship with the differential gene may be determined as a regulatory gene of the differential gene based on the regulatory relationship network between the target genes.

The differential gene is a gene that is determined from the target genes and that has different expression in different cells. For example, the material group data of the target gene includes expression values of the target gene in a plurality of cells. The cells may be cells of a cell type from tissues having different disease states (for example, a cancerous tissue and a para-cancerous tissue). Information about whether the target gene is the differential gene may be obtained by performing differential gene analysis on the material group data of the target gene. For example, a mean squared error, a standard deviation, and another error of the expression values of the target gene in the plurality of cells are calculated to obtain an error of the target gene. When the error of the target gene is greater than a set error, the target gene is the differential gene.

After the regulatory gene of the differential gene is determined, products expressed by the regulatory gene may be determined (in other words, a gene function is determined). The products expressed by the regulatory gene includes an RNA and a protein. A disease may be analyzed based on the products expressed by the regulatory gene, to facilitate treatment for the disease. For example, the protein expressed by the regulatory gene is used as a candidate drug target, and a target drug target is determined from the candidate drug target by experimental verification. The target drug target facilitates binding of small drug molecules and drug discovery, so as to facilitate the treatment of disease.

Information (including but not limited to user equipment information, user personal information, and the like), data (including but not limited to data used for analysis, stored data, displayed data, and the like), and a signal included in this application are authorized by a user or fully authorized by parties. Collection, use and processing of relevant data shall comply with relevant laws, regulations, and standards of relevant countries and regions. For example, the material group data of the target gene and the annotated regulatory relationship between the target gene pair included in this application are obtained under full authorization.

In the foregoing method, the predicted regulatory relationship between the two target genes among the plurality of target genes is determined by using the gene regulatory relationship detection model based on the material group data of the plurality of target genes. In addition, based on the predicted regulatory relationship between the two target genes, it is determined whether the regulatory relationship exists between the two target genes. The gene regulatory relationship detection model is used to detect whether the regulatory relationship exists between the two target genes, to facilitate understanding a mechanism of disease occurrence and development at a level of gene regulation.

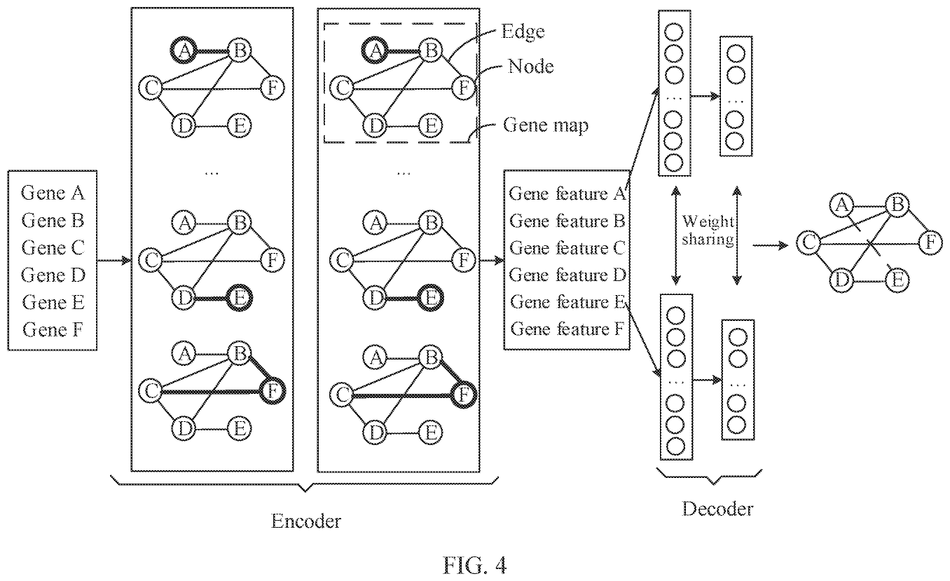

From a perspective of method steps, the gene regulatory relationship detection model training method and the regulatory relationship detection method according to embodiments of this application are described above. The following is further described with reference to FIG. 4. FIG. 4 is a schematic diagram of detecting a regulatory relationship according to an embodiment of this application. A neural network model includes both an encoder and a decoder shown in FIG. 4. A gene regulatory relationship detection model is obtained by training the neural network model. The gene regulatory relationship detection model may be used to detect whether a regulatory relationship exists between target genes.

Sample genes may be used to train the gene regulatory relationship detection model. For example, the sample genes are represented by a gene A to a gene F respectively. Material group data of the gene A to material group data of the gene F are used as a node A to a node F respectively. In addition, a reported regulatory relationship between genes may be obtained. For example, a regulatory relationship exists between the gene A that is reported and the gene B. Therefore, an edge is added between the node A and the node B. Regulatory relationships exist between the gene B that is reported, the gene A, a gene C, a gene D, and the gene F. Therefore, edges are added between the node B, the node A, a node C, a node D, and the node F. By analogy, a gene map may be obtained.

For any node, when the encoder determines, based on the gene map, that an adjacent node of the node exists, the node is updated based on the node and the adjacent node. When the encoder determines, based on the gene map, that no adjacent node of the node exists, the node is updated based on the node. An edge exists between the node and the adjacent node of the node. For example, when an adjacent node of the node A is the node B, the node A is updated based on the node A and the node B. When adjacent nodes of the node F are the node B and the node C, the node F is updated based on the node B, the node C, and the node F, and so on. The encoder may update each node many times, and a node obtained by the last update is a gene feature of a gene corresponding to the node. For example, the node A obtained by the last update is a gene feature of a gene A (referred to as a gene feature A), and the node B obtained by the last update is a gene feature of a gene B (referred to as a gene feature B).

For any two gene features, the decoder may perform dimension reduction on the two gene features. Two gene features on which dimension reduction is performed are used to determine a probability that a regulatory relationship exists between two genes, so that it is determined whether the regulatory relationship exists the two genes. For example, while dimension reduction is performed on the gene feature A, dimension reduction is performed on a gene feature E, and weight is shared when dimension reduction is performed on the gene feature A and the gene feature E. In other words, the same decoder is used to perform dimension reduction on the gene feature A and the gene feature E. Then, a probability that an edge exists between the node A and the node E is determined by using a gene feature A on which dimension reduction is performed and a gene feature E on which dimension reduction is performed. Because that an edge exists between nodes represents that a regulatory relationship exists between genes corresponding to the nodes, the decoder may determine and output a probability that a regulatory relationship exists between the gene A and the gene E, so that it is determined whether the regulatory relationship exists between the gene A and the gene E.

The encoder and the decoder included in the neural network model may be trained by using a probability that a regulatory relationship exists between each two genes and a regulatory relationship between reported genes, to obtain the gene regulatory relationship detection model. The gene regulatory relationship detection model may be configured to determine whether a regulatory relationship exists between each two target genes. A method in which the gene regulatory relationship detection model determines whether the regulatory relationship exists between the two target genes is similar to a method in which the neural network model determines whether a regulatory relationship exists between two genes. Details are not described herein again.

By training the gene regulatory relationship detection model, the gene regulatory relationship detection model may be used to detect whether the regulatory relationship exists between the two target genes, to facilitate understanding a mechanism of disease occurrence and development at a level of gene regulation.

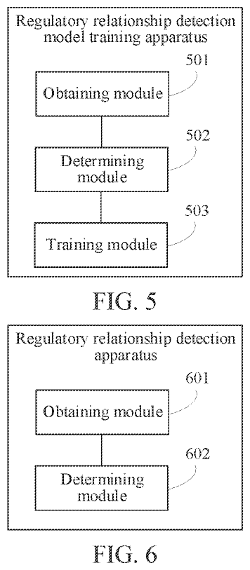

FIG. 5 shows a schematic diagram of a structure of a gene regulatory relationship detection model training apparatus according to an embodiment of this application. As shown in FIG. 5, the apparatus includes:
an obtaining module 501, configured to obtain material group data of a plurality of sample genes and an annotated regulatory relationship between at least one sample gene pair, an annotated regulatory relationship between any sample gene pair being information about that a regulatory relationship exists between two sample genes in the sample gene pair obtained by annotation (in other words, configured to obtain material group data of a plurality of sample genes and an annotated regulatory relationship between at least one sample gene pair obtained by annotation, an annotated regulatory relationship between any sample gene pair indicating that a regulatory relationship exists between two sample genes in the sample gene pair);
a determining module 502, configured to determine a predicted regulatory relationship between each two sample genes among the plurality of sample genes by using a neural network model based on the material group data of the plurality of sample genes, a predicted regulatory relationship between any two sample genes being a probability that a regulatory relationship exists between the two sample genes, obtained by prediction (in other words, configured to determine a predicted probability between each two sample genes among the plurality of sample genes by using a neural network model based on the material group data of the plurality of sample genes, a predicted probability between any two sample genes being a probability that a regulatory relationship exists between the two sample genes, obtained by prediction); and
a training module 503, configured to train the neural network model to obtain a gene regulatory relationship detection model, based on the annotated regulatory relationship between the at least one sample gene pair and the predicted regulatory relationship between each two sample genes, the gene regulatory relationship detection model being configured to detect whether a regulatory relationship exists between two target genes.

In a possible implementation, the determining module 502 is configured to: perform feature extraction on the material group data of the plurality of sample genes by using the neural network model based on the annotated regulatory relationship between the at least one sample gene pair, to obtain a gene feature of each sample gene; and determine the predicted regulatory relationship (that is, the predicted probability) between each two sample genes among the plurality of sample genes by using the neural network model based on a gene feature of each sample gene.

In a possible implementation, the determining module 502 is configured to: for any sample gene, determine a gene feature of the sample gene by using the neural network model based on material group data of the sample gene and material group data of an adjacent gene of the sample gene, when it is determined, based on the annotated regulatory relationship between the at least one sample gene pair, that the adjacent gene of the sample gene exists, the adjacent gene of the sample gene being a sample gene that has a regulatory relationship with the sample gene; or determine a gene feature of the sample gene by using the neural network model based on material group data of the sample gene, when it is determined, based on the annotated regulatory relationship between the at least one sample gene pair, that no adjacent gene of the sample gene exists.

In a possible implementation, the determining module 502 is configured to: determine each sample node based on material group data of each sample gene, one sample node representing material group data of one sample gene; for any two sample nodes, add a sample edge between the two sample nodes, when it is determined, based on the annotated regulatory relationship between the at least one sample gene pair, that a regulatory relationship exists between sample genes corresponding to the two sample nodes; and perform feature extraction on a sample gene map by using the neural network model to obtain the gene feature of each sample gene, the sample gene map including each sample node and the sample edge.

In a possible implementation, the determining module 502 is configured to: for any sample node, determine a gene feature of a sample gene corresponding to the sample node based on the sample node and an adjacent node of the sample node, when it is determined, based on the sample gene map, that the adjacent node of the sample node exists, the adjacent node of the sample node being a sample node that has a sample edge with the sample node; or determine a gene feature of a sample gene corresponding to the sample node based on the sample node, when it is determined, based on the sample gene map, that no adjacent node of the sample node exists.

In a possible implementation, the determining module 502 is configured to: for any two sample genes among the plurality of sample genes, determine a degree of similarity between gene features of the two sample genes by using the neural network model; and determine the predicted regulatory relationship (that is, the predicted probability) between the two sample genes based on the degree of similarity between the gene features of the two sample genes.

In a possible implementation, the determining module 502 is configured to: for the two sample genes among the plurality of sample genes, determine a sample scatter plot based on material group data of the two sample genes, material group data of any sample gene including expression values of the sample gene in a plurality of cells, the sample scatter plot including a plurality of sample points, and any sample point representing expression values of the two sample genes in a same cell; perform feature extraction on the sample scatter plot by using the neural network model to obtain sample image features; and classify the sample image features by using the neural network model, and use a classification result as the predicted regulatory relationship (that is, the predicted probability) between the two sample genes.

In a possible implementation, the determining module 502 is configured to: divide the sample scatter plot into a plurality of sample image regions; and perform the feature extraction on the sample scatter plot by using the neural network model based on a quantity of sample points in each sample image region to obtain the sample image features.

In a possible implementation, the training module 503 is configured to: for the two sample genes, determine a reference regulatory relationship between the two sample genes based on the predicted regulatory relationship between the two sample genes, the reference regulatory relationship between the two sample genes being information about whether the regulatory relationship exists between the two sample genes obtained by prediction (in other words, for the two sample genes, obtain a reference regulatory relationship between the two sample genes based on the predicted probability between the two sample genes by prediction, the reference regulatory relationship between the two sample genes indicating whether the regulatory relationship exists between the two sample genes); and train the neural network model to obtain the gene regulatory relationship detection model, based on the annotated regulatory relationship between the at least one sample gene pair and the reference regulatory relationship between the two sample genes.

FIG. 6 shows a schematic diagram of a structure of a regulatory relationship detection apparatus according to an embodiment of this application. As shown in FIG. 6, the apparatus may include:
an obtaining module 601, configured to obtain material group data of a plurality of target genes and a gene regulatory relationship detection model, the gene regulatory relationship detection model being obtained by training according to any of the foregoing gene regulatory relationship detection model training method; and
a determining module 602, configured to determine a predicted regulatory relationship (that is, a predicted probability) between each two target genes among the plurality of target genes by using the gene regulatory relationship detection model based on the material group data of the plurality of target genes, a predicted regulatory relationship (that is, a predicted probability) between any two target genes being a probability that a regulatory relationship exists between the two target genes, obtained by prediction.

The determining module 602 is further configured to, for the two target genes, determine, based on the probability that the regulatory relationship exists between the two target genes, whether the regulatory relationship exists between the two target genes.

In a possible implementation, the determining module 602 is configured to: obtain an annotated regulatory relationship between at least one target gene pair, an annotated regulatory relationship between any target gene pair being information about that a regulatory relationship exists between two target genes in the target gene pair, obtained by annotation (in other words, configured to obtain an annotated regulatory relationship between at least one target gene pair obtained by annotation, an annotated regulatory relationship between any target gene pair indicating that a regulatory relationship exists between two target genes in the target gene pair); perform feature extraction on the material group data of the plurality of target genes by using the gene regulatory relationship detection model based on the annotated regulatory relationship between the at least one target gene pair, to obtain a gene feature of each target gene; and determine the predicted regulatory relationship (that is, the predicted probability) between the two target genes by using the gene regulatory relationship detection model based on the gene feature of each target gene.

In a possible implementation, the determining module 602 is configured to: for the two target genes, determine a target scatter plot based on material group data of the two target genes, material group data of any target gene including expression values of the target gene in a plurality of cells, the target scatter plot including a plurality of target points, and any target point representing expression values of the two target genes in a same cell; perform feature extraction on the target scatter plot by using the gene regulatory relationship detection model to obtain target image features; and classify the target image features by using the gene regulatory relationship detection model to obtain the predicted regulatory relationship (that is, the predicted probability) between the two target genes.

In a possible implementation, the determining module 602 is configured to: determine, when the probability that the regulatory relationship exists between the two target genes is greater than a threshold, that the regulatory relationship exists between the two target genes; or determine, when the probability that the regulatory relationship exists between the two target genes is not greater than a threshold, that no regulatory relationship exists between the two target genes.

When the apparatus provided in FIG. 5 or FIG. 6 implements functions of the apparatus, only division of the foregoing function modules is used as an example for description. In the practical application, the functions may be allocated to and completed by different function modules according to requirements. That is, an internal structure of the device is divided into different function modules, to complete all or some of the functions described above. In addition, the apparatus provided in the foregoing embodiments and the method embodiments are fall within the same concept. For a specific implementation process and the produced technical effects, refer to the foregoing method embodiments. Details are not described herein again.

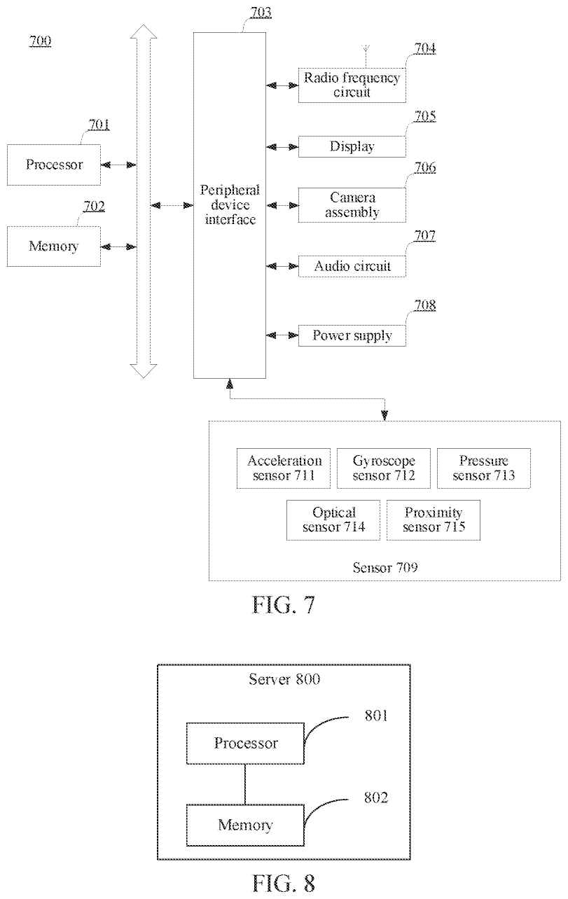

FIG. 7 is a block diagram of a structure of a terminal device 700 according to an exemplary embodiment of this application. The terminal device 700 includes a processor 701 and a memory 702.

The processor 701 may include one or more processing cores, for example, a 4-core processor or an 8-core processor. The processor 701 may be implemented in at least one hardware form of a digital signal processing (DSP), a field programmable gate array (FPGA), and a programmable logic array (PLA). The processor 701 may also include a main processor and a coprocessor. The main processor is a processor configured to process data in an awake state, and is also referred to as a central processing unit (CPU). The coprocessor is a low power consumption processor configured to process the data in a standby state. In some embodiments, the processor 701 may be integrated with a graphics processing unit (GPU). The GPU is configured to render and draw content that needs to be displayed on a display. In some embodiments, the processor 701 may further include an artificial intelligence (AI) processor. The AI processor is configured to process computing operations related to machine learning.

The memory 702 may include one or more computer-readable storage medium. The computer-readable storage medium may be non-transient (or non-temporary). The memory 702 may further include a high-speed random access memory and a nonvolatile memory, for example, one or more disk storage devices or flash storage devices. In some embodiments, the non-transitory computer-readable storage medium in the memory 702 is configured to store at least one computer program, and the at least one computer program is configured to be executed by the processor 701 to implement the gene regulatory relationship detection model training method or the regulatory relationship detection method provided in the method embodiments of this application.

In some embodiments, the terminal device 700 may alternatively include a peripheral device interface 703 and at least one peripheral device. The processor 701, the memory 702, and the peripheral device interface 703 may be connected via a bus or a signal cable. Each peripheral device may be connected to the peripheral device interface 703 via a bus, a signal cable, or a circuit board. Specifically, the peripheral device includes at least one of a radio frequency circuit 704, a display 705, a camera assembly 706, an audio circuit 707, and a power supply 708.

The peripheral device interface 703 may be configured to connect the at least one peripheral device related to input/output (I/O) to the processor 701 and the memory 702. In some embodiments, the processor 701, the memory 702, and the peripheral device interface 703 are integrated on the same chip or circuit board. In some other embodiments, any one or two of the processor 701, the memory 702, and the peripheral device interface 703 may be implemented on a single chip or circuit board. This is not limited in this embodiment.

The radio frequency circuit 704 is configured to receive and transmit a radio frequency (RF) signal, also referred to as an electromagnetic signal. The radio frequency circuit 704 communicates with a communication network and another communication device by using the electromagnetic signal. The radio frequency circuit 704 converts an electric signal into an electromagnetic signal for transmission, or converts a received electromagnetic signal into an electric signal. In one embodiment, the radio frequency circuit 704 includes an antenna system, an RF transceiver, one or more amplifiers, a tuner, an oscillator, a digital signal processor, a codec chip set, a subscriber identity module card, and the like. The radio frequency circuit 704 may communicate with other terminals by using at least one wireless communication protocol. The wireless communication protocol includes, but is not limited to: a world wide web, a metropolitan area network, an intranet, generations of mobile communication networks (2G, 3G, 4G, and 5G), a wireless local area network and/or a wireless fidelity (Wi-Fi) network. In some embodiments, the radio frequency circuit 704 may further include a circuit related to near field communication (NFC). This is not limited in this application.

The display 705 is configured to display a user interface (UI). The UI may include a graph, text, an icon, a video, and any combination thereof. When the display 705 is a touch display, the display 705 also has a capability to collect a touch signal on or above a surface of the display 705. The touch signal may be inputted to the processor 701 as a control signal for processing. In this case, the display 705 may be further configured to provide a virtual button and/or a virtual keyboard, also referred to as a soft button and/or a soft keyboard. In some embodiments, there may be one display 705 disposed on a front panel of the terminal device 700. In other embodiments, there may be at least two displays 705 disposed on different surfaces of the terminal device 700 respectively or in a folded design. In other embodiments, the display 705 may be a flexible display disposed on a curved or folded surface of the terminal device 700. Even, the display 705 may be further set in a non-rectangular irregular pattern, namely, a special-shaped screen. The display 705 may be prepared by using materials such as a liquid crystal display (LCD) or an organic light-emitting diode (OLED).

The camera assembly 706 is configured to collect images or videos. In one embodiment, the camera assembly 706 includes a front-facing camera and a rear-facing camera. Generally, the front-facing camera is disposed on the front panel of the terminal, and the rear-facing camera is disposed on a back surface of the terminal. In some embodiments, there are at least two rear-facing cameras, which are respectively any of a main camera, a depth-of-field camera, a wide-angle camera, and a telephoto camera, to achieve background blur through fusion of the main camera and the depth-of-field camera, panoramic photographing and virtual reality (VR) photographing through fusion of the main camera and the wide-angle camera, or other fusion photographing functions. In some embodiments, the camera assembly 706 may further include a flash. The flash may be a monochrome temperature flash, or may be a double color temperature flash. The double color temperature flash refers to a combination of a warm light flash and a cold light flash, and may be used for light compensation under different color temperatures.

The audio circuit 707 may include a microphone and a speaker. The microphone is configured to collect sound waves of a user and an environment, and convert the sound waves into an electrical signal to input to the processor 701 for processing, or input to the radio frequency circuit 704 for implementing voice communication. For a purpose of stereo collection or noise reduction, there may be a plurality of microphones disposed at different portions of the terminal device 700 respectively. The microphone may further be an array microphone or an omni-directional collection type microphone. The speaker is configured to convert an electrical signal from the processor 701 or the radio frequency circuit 704 into sound waves. The speaker may be a conventional film speaker, or may be a piezoelectric ceramic speaker. When the speaker is the piezoelectric ceramic speaker, the speaker not only can convert an electric signal into sound waves audible to a human being, but also can convert an electric signal into sound waves inaudible to a human being, for ranging and other purposes. In some embodiments, the audio circuit 707 may further include an earphone jack.

The power supply 708 is configured to supply power to components in the terminal device 700. The power supply 708 may be an alternating current, a direct current, a disposable battery, or a rechargeable battery. When the power supply 708 includes a rechargeable battery, and the rechargeable battery may be a wired rechargeable battery or a wireless rechargeable battery. The wired rechargeable battery is a battery charged through a wired circuit, and the wireless rechargeable battery is a battery charged through a wireless coil. The rechargeable battery may be further configured to support a fast charging technology.

In some embodiments, the terminal device 700 further includes one or more sensors 709. The one or more sensors 709 include, but are not limited to: an acceleration sensor 711, a gyroscope sensor 712, a pressure sensor 713, an optical sensor 714, and a proximity sensor 715.

The acceleration sensor 711 may measure a magnitude of acceleration on three coordinate axes of a coordinate system established with the terminal device 700. For example, the acceleration sensor 711 may be configured to measure components of gravity acceleration on the three coordinate axes. The processor 701 may control, according to a gravity acceleration signal collected by the acceleration sensor 711, the display 705 to display the user interface in a transverse view or a longitudinal view. The acceleration sensor 711 may be further configured to collect data of a game or a user movement.

The gyroscope sensor 712 may measure a body direction and a rotation angle of the terminal device 700. The gyroscope sensor 712 may cooperate with the acceleration sensor 711 to collect a 3D action by the user on the terminal device 700. The processor 701 may implement the following functions according to the data collected by the gyroscope sensor 712: motion sensing (such as changing the UI according to a tilt operation performed by the user), image stabilization during shooting, game control, and inertial navigation.

The pressure sensor 713 may be disposed at a side frame of the terminal device 700 and/or a lower layer of the display 705. When the pressure sensor 713 is disposed on the side frame of the terminal device 700, a holding signal of the user on the terminal device 700 may be detected. The processor 701 performs left and right hand recognition or a quick operation based on the holding signal collected by the pressure sensor 713. When the pressure sensor 713 is disposed on the lower layer of the display 705, the processor 701 controls an operable control on the UI based on a pressure operation performed by the user on the display 705. The operable control includes at least one of a button control, a scroll-bar control, an icon control, and a menu control.

The optical sensor 714 is configured to collect ambient light intensity. In an embodiment, the processor 701 may control display brightness of the display 705 according to the ambient light intensity collected by the optical sensor 714. Specifically, when the ambient light intensity is high, the display brightness of the display 705 is increased. When the ambient light intensity is low, the display brightness of the display 705 is decreased. In another embodiment, the processor 701 may further dynamically adjust a camera parameter of the camera assembly 706 according to the ambient light intensity collected by the optical sensor 714.

The proximity sensor 715, also referred to as a distance sensor, is generally disposed on the front panel of the terminal device 700. The proximity sensor 715 is configured to collect a distance between the user and a front surface of the terminal device 700. In an embodiment, when the proximity sensor 715 detects that the distance between the user and the front surface of the terminal device 700 gradually decreases, the display 705 is controlled by the processor 701 to switch from a screen-on state to a screen-off state. When the proximity sensor 715 detects that the distance between the user and the front surface of the terminal device 700 gradually increases, the display 705 is controlled by the processor 701 to switch from the screen-off state to the screen-on state.

A person skilled in the art may understand that the structure shown in FIG. 7 does not constitute a limitation on the terminal device 700, and the terminal device may include more or fewer components than those shown in the figure, or some components may be combined, or a different component deployment may be used.

FIG. 8 is a schematic diagram of a structure of a server according to an embodiment of this application. The server 800 may vary a lot due to different configurations or performance, and may include one or more processors 801 and one or more memories 802. The one or more memories 802 have at least one computer program stored thereon, the at least one computer program being loaded and executed by the one or more processors 801 to implement the gene regulatory relationship detection model training method and the regulatory relationship detection method provided in the foregoing method embodiments. For example, the processor 801 is a CPU. Certainly, the server 800 may further include components such as a wired or wireless network interface, a keyboard, and an input/output interface, to facilitate input and output. The server 800 may further include another component configured to implement a function of a device. Details are not described herein again.

In an exemplary embodiment, a non-transitory computer-readable storage medium is provided, the non-transitory computer-readable storage medium has at least one computer program stored thereon, and the at least one computer program is loaded and executed by a processor to enable an electronic device to implement the foregoing gene regulatory relationship detection model training method or the regulatory relationship detection method.

In one embodiment, the foregoing non-transitory computer-readable storage medium may be a read-only memory (ROM), a random access memory (RAM), a compact disc read-only memory (CD-ROM), a magnetic tape, a floppy disk, an optical data storage device, or the like.

In an exemplary embodiment, a computer program or a computer program product is further provided, the computer program or the computer program product has at least one computer program stored thereon, and the at least one computer program is loaded and executed by a processor to enable an electronic device to implement the foregoing gene regulatory relationship detection model training method or the regulatory relationship detection method.

In this application, the term “module” in this application refers to a computer program or part of the computer program that has a predefined function and works together with other related parts to achieve a predefined goal and may be all or partially implemented by using software, hardware (e.g., processing circuitry and/or memory configured to perform the predefined functions), or a combination thereof. Each module can be implemented using one or more processors (or processors and memory). Likewise, a processor (or processors and memory) can be used to implement one or more modules. Moreover, each module can be part of an overall module that includes the functionalities of the module. The term “plurality of” mentioned in the specification means two or more. The term “and/or” describes an association relationship between associated objects and indicates that three relationships may exist. For example, A and/or B may indicate the following three cases: Only A exists, both A and B exist, and only B exists. The character “/” generally indicates an “or” relationship between the associated objects.

The sequence numbers of the foregoing embodiments of this application are merely for description purpose but do not imply the preference among the embodiments.

The foregoing description is merely exemplary embodiments of this application, but is not intended to limit this application. Any modification, equivalent replacement, or improvement made within the principle of this application shall fall within the protection scope of this application.

What is claimed is:

1. A method for training a gene regulatory relationship detection model performed by an electronic device, and the method comprising:
obtaining material group data of a plurality of sample genes and an annotated regulatory relationship between at least one sample gene pair indicating that a regulatory relationship exists between two sample genes in the sample gene pair;
determining a predicted probability that a regulatory relationship exists between each two sample genes among the plurality of sample genes by using a neural network model based on the material group data of the plurality of sample genes; and
training the neural network model to obtain a gene regulatory relationship detection model, based on the annotated regulatory relationship between the at least one sample gene pair and the predicted probability between each two sample genes.

2. The method according to claim 1, wherein the determining a predicted probability that a regulatory relationship exists between each two sample genes among the plurality of sample genes by using a neural network model based on the material group data of the plurality of sample genes comprises:
performing feature extraction on the material group data of the plurality of sample genes by using the neural network model based on the annotated regulatory relationship between the at least one sample gene pair, to obtain a gene feature of each sample gene; and
determining the predicted probability that a regulatory relationship exists between each two sample genes among the plurality of sample genes by using the neural network model based on the gene feature of each sample gene.

3. The method according to claim 2, wherein the performing feature extraction on the material group data of the plurality of sample genes by using the neural network model based on the annotated regulatory relationship between the at least one sample gene pair, to obtain a gene feature of each sample gene comprises:
determining a gene feature of a sample gene by using the neural network model based on material group data of the sample gene and material group data of an adjacent gene of the sample gene, when it is determined, based on the annotated regulatory relationship between the at least one sample gene pair, that the adjacent gene of the sample gene exists, the adjacent gene of the sample gene being a sample gene that has a regulatory relationship with the sample gene; or
determining a gene feature of the sample gene by using the neural network model based on material group data of the sample gene, when it is determined, based on the annotated regulatory relationship between the at least one sample gene pair, that no adjacent gene of the sample gene exists.

4. The method according to claim 2, wherein the performing feature extraction on the material group data of the plurality of sample genes by using the neural network model based on the annotated regulatory relationship between the at least one sample gene pair, to obtain a gene feature of each sample gene comprises:
determining each sample node based on material group data of each sample gene, one sample node representing material group data of one sample gene;
adding a sample edge between two sample nodes, when it is determined, based on the annotated regulatory relationship between the at least one sample gene pair, that a regulatory relationship exists between sample genes corresponding to the two sample nodes; and
performing feature extraction on a sample gene map by using the neural network model to obtain the gene feature of each sample gene, the sample gene map comprising each sample node and the sample edge.

5. The method according claim 2, wherein the determining the predicted probability that a regulatory relationship exists between each two sample genes among the plurality of sample genes by using the neural network model based on the gene feature of each sample gene comprises:
for two sample genes among the plurality of sample genes, determining a degree of similarity between gene features of the two sample genes by using the neural network model; and
determining the predicted probability between the two sample genes based on the degree of similarity between the gene features of the two sample genes.

6. The method according to claim 1, wherein the determining a predicted probability that a regulatory relationship exists between each two sample genes among the plurality of sample genes by using a neural network model based on the material group data of the plurality of sample genes comprises:
for the two sample genes among the plurality of sample genes, determining a sample scatter plot based on material group data of the two sample genes, material group data of any sample gene comprising expression values of the sample gene in a plurality of cells, the sample scatter plot comprising a plurality of sample points, and any sample point representing expression values of the two sample genes in a same cell;
performing feature extraction on the sample scatter plot by using the neural network model to obtain sample image features; and
classifying the sample image features by using the neural network model, and using a classification result as the predicted probability between the two sample genes.

7. The method according to claim 1, wherein the training the neural network model to obtain a gene regulatory relationship detection model, based on the annotated regulatory relationship between the at least one sample gene pair and the predicted probability between each two sample genes comprises:
for the two sample genes, obtaining a reference regulatory relationship between the two sample genes based on the predicted probability between the two sample genes by prediction, the reference regulatory relationship between the two sample genes indicating whether the regulatory relationship exists between the two sample genes; and
training the neural network model to obtain the gene regulatory relationship detection model, based on the annotated regulatory relationship between the at least one sample gene pair and the reference regulatory relationship between the two sample genes.

8. The method according to claim 1, wherein the method further comprises:
obtaining material group data of a plurality of target genes;
determining a predicted probability that a regulatory relationship exists between each two target genes among the plurality of target genes by using the gene regulatory relationship detection model based on the material group data of the plurality of target genes.

9. An electronic device, comprising a processor and a memory, the memory having at least one computer program stored thereon, and the at least one computer program being loaded and executed by the processor to enable the electronic device to implement the gene regulatory relationship detection model training method including:
obtaining material group data of a plurality of sample genes and an annotated regulatory relationship between at least one sample gene pair indicating that a regulatory relationship exists between two sample genes in the sample gene pair;
determining a predicted probability that a regulatory relationship exists between each two sample genes among the plurality of sample genes by using a neural network model based on the material group data of the plurality of sample genes; and
training the neural network model to obtain a gene regulatory relationship detection model, based on the annotated regulatory relationship between the at least one sample gene pair and the predicted probability between each two sample genes.

10. The electronic device according to claim 9, wherein the determining a predicted probability that a regulatory relationship exists between each two sample genes among the plurality of sample genes by using a neural network model based on the material group data of the plurality of sample genes comprises:
performing feature extraction on the material group data of the plurality of sample genes by using the neural network model based on the annotated regulatory relationship between the at least one sample gene pair, to obtain a gene feature of each sample gene; and
determining the predicted probability that a regulatory relationship exists between each two sample genes among the plurality of sample genes by using the neural network model based on the gene feature of each sample gene.

11. The electronic device according to claim 10, wherein the determining the predicted probability that a regulatory relationship exists between each two sample genes among the plurality of sample genes by using the neural network model based on the gene feature of each sample gene comprises:
for two sample genes among the plurality of sample genes, determining a degree of similarity between gene features of the two sample genes by using the neural network model; and
determining the predicted probability between the two sample genes based on the degree of similarity between the gene features of the two sample genes.

12. The electronic device according to claim 9, wherein the determining a predicted probability that a regulatory relationship exists between each two sample genes among the plurality of sample genes by using a neural network model based on the material group data of the plurality of sample genes comprises:
for the two sample genes among the plurality of sample genes, determining a sample scatter plot based on material group data of the two sample genes, material group data of any sample gene comprising expression values of the sample gene in a plurality of cells, the sample scatter plot comprising a plurality of sample points, and any sample point representing expression values of the two sample genes in a same cell;
performing feature extraction on the sample scatter plot by using the neural network model to obtain sample image features; and
classifying the sample image features by using the neural network model, and using a classification result as the predicted probability between the two sample genes.

13. The electronic device according to claim 9, wherein the training the neural network model to obtain a gene regulatory relationship detection model, based on the annotated regulatory relationship between the at least one sample gene pair and the predicted probability between each two sample genes comprises:
for the two sample genes, obtaining a reference regulatory relationship between the two sample genes based on the predicted probability between the two sample genes by prediction, the reference regulatory relationship between the two sample genes indicating whether the regulatory relationship exists between the two sample genes; and
training the neural network model to obtain the gene regulatory relationship detection model, based on the annotated regulatory relationship between the at least one sample gene pair and the reference regulatory relationship between the two sample genes.

14. The electronic device according to claim 9, wherein the method further comprises:
obtaining material group data of a plurality of target genes;
determining a predicted probability that a regulatory relationship exists between each two target genes among the plurality of target genes by using the gene regulatory relationship detection model based on the material group data of the plurality of target genes.

15. A non-transitory computer-readable storage medium, having at least one computer program stored thereon, and the at least one computer program being loaded and executed by a processor to enable an electronic device to implement a gene regulatory relationship detection model training method including:
obtaining material group data of a plurality of sample genes and an annotated regulatory relationship between at least one sample gene pair indicating that a regulatory relationship exists between two sample genes in the sample gene pair;
determining a predicted probability that a regulatory relationship exists between each two sample genes among the plurality of sample genes by using a neural network model based on the material group data of the plurality of sample genes; and
training the neural network model to obtain a gene regulatory relationship detection model, based on the annotated regulatory relationship between the at least one sample gene pair and the predicted probability between each two sample genes.

16. The non-transitory computer-readable storage medium according to claim 15, wherein the determining a predicted probability that a regulatory relationship exists between each two sample genes among the plurality of sample genes by using a neural network model based on the material group data of the plurality of sample genes comprises:
performing feature extraction on the material group data of the plurality of sample genes by using the neural network model based on the annotated regulatory relationship between the at least one sample gene pair, to obtain a gene feature of each sample gene; and
determining the predicted probability that a regulatory relationship exists between each two sample genes among the plurality of sample genes by using the neural network model based on the gene feature of each sample gene.

17. The non-transitory computer-readable storage medium according to claim 16, wherein the determining the predicted probability that a regulatory relationship exists between each two sample genes among the plurality of sample genes by using the neural network model based on the gene feature of each sample gene comprises:
for two sample genes among the plurality of sample genes, determining a degree of similarity between gene features of the two sample genes by using the neural network model; and
determining the predicted probability between the two sample genes based on the degree of similarity between the gene features of the two sample genes.

18. The non-transitory computer-readable storage medium according to claim 15, wherein the determining a predicted probability that a regulatory relationship exists between each two sample genes among the plurality of sample genes by using a neural network model based on the material group data of the plurality of sample genes comprises:
for the two sample genes among the plurality of sample genes, determining a sample scatter plot based on material group data of the two sample genes, material group data of any sample gene comprising expression values of the sample gene in a plurality of cells, the sample scatter plot comprising a plurality of sample points, and any sample point representing expression values of the two sample genes in a same cell;
performing feature extraction on the sample scatter plot by using the neural network model to obtain sample image features; and
classifying the sample image features by using the neural network model, and using a classification result as the predicted probability between the two sample genes.

19. The non-transitory computer-readable storage medium according to claim 15, wherein the training the neural network model to obtain a gene regulatory relationship detection model, based on the annotated regulatory relationship between the at least one sample gene pair and the predicted probability between each two sample genes comprises:
for the two sample genes, obtaining a reference regulatory relationship between the two sample genes based on the predicted probability between the two sample genes by prediction, the reference regulatory relationship between the two sample genes indicating whether the regulatory relationship exists between the two sample genes; and
training the neural network model to obtain the gene regulatory relationship detection model, based on the annotated regulatory relationship between the at least one sample gene pair and the reference regulatory relationship between the two sample genes.

20. The non-transitory computer-readable storage medium according to claim 15, wherein the method further comprises:
obtaining material group data of a plurality of target genes;
determining a predicted probability that a regulatory relationship exists between each two target genes among the plurality of target genes by using the gene regulatory relationship detection model based on the material group data of the plurality of target genes.
